(19) United States
(12) Patent Application Publication
(10) Pub. No.: US 2018/0055451 A1
(43) Pub. Date: Mar. 1, 2018

(54) BIOLOGICAL INFORMATION DETECTION APPARATUS

(71) Applicants: Shinichi Kuroyanagi, Aichi (JP); Tadao Nishiguchi, Aichi (JP)

(72) Inventors: Shinichi Kuroyanagi, Aichi (JP); Tadao Nishiguchi, Aichi (JP)

(73) Assignee: OMRON AUTOMOTIVE ELECTRONICS CO., LTD., Aichi (JP)

(21) Appl. No.: 15/684,547

(22) Filed: Aug. 23, 2017

(30) Foreign Application Priority Data
Aug. 24, 2016 (JP) 2016-163703

(51) Int. Cl. A61B 5/00; G01S 13/88; G01S 13/56

(52) U.S. Cl. CPC A61B 5/7207; A61B 5/7225; A61B 5/024; G01S 13/88; G01S 13/56; A61B 5/725

(57) ABSTRACT

A biological information detection apparatus includes a vehicle opening detector, a riding detector, a radio wave transmitter-receiver, a biological information detector, and a correction value setting unit. The radio wave transmitter-receiver transmits a radio wave and receive a reflected wave of the radio wave, and the correction value setting unit sets a correction value for detecting biological information based on the receiving result after detection of opening of a vehicle by the vehicle opening detector and before detection of riding of a driver in the vehicle by the riding detector. The radio wave transmitter-receiver transmits a radio wave and receives a reflected wave reflected by the body surface of the driver, and the biological information detector detects biological information of the driver based on the receiving result and the correction value after the detection of riding of the driver in the vehicle by the riding detector.

### CROSS-REFERENCE TO RELATED APPLICATION

This application is based on Japanese Patent Application No. 2016-163703 filed with the Japan Patent Office on Aug. 24, 2016, the entire contents of which are incorporated herein by reference.

### FIELD

The disclosure relates to a biological information detection apparatus that transmits and receives a radio wave to and from a body surface of an occupant of a vehicle to detect biological information of the occupant.

### BACKGROUND

For example, JP 2013-153782 A, JP 2015-89513 A, JP 2015-97638 A, JP 2006-264464 A, JP 2006-55504 A, JP 2010-120493 A, JP 2011-15887 A, and JP 10-179527 A disclose techniques for detecting biological information of an occupant of a vehicle for preventing a vehicle accident in advance.

In JP 2013-153782 A, a heart rate sensor detects heart rate signals of a subject. Then, among the heart rate signals output from the heart rate sensor, a signal having a frequency equal to or lower than a predetermined first frequency is allowed to pass through a low-pass filter and subjected to high frequency compensation to acquire a harmonic signal of low frequency noise. Further, among the heart rate signals output from the heart rate sensor, a signal having a frequency equal to or higher than a predetermined second frequency is allowed to pass through a high-pass filter to remove a harmonic signal of low frequency noise from the signal. Accordingly, respiration signals are removed from the heart rate signals detected by the heart rate sensor to acquire an excellent heart rate sensing characteristic.

In JP 2015-89513 A, a multidimensional sensor array is disposed at a position for sensing biological information of a human. A plurality of sensors included in the multidimensional sensor array are mechanically coupled to a common structural coupling material. A biological signal is output based on selectively receiving an output from each of the sensors and processing the output from each of the sensors. Accordingly, the biological signal is acquired inside a vehicle without interferences by noise or vibrations from an engine of the vehicle and a road travel amount.

In JP 2015-97638 A, two IQ orthogonal signals (VI (t), VQ (t)) which are output by a Doppler radar module at a time t are subjected to approximation by a least squares method using an N+1 polynomial expression (N is a natural number of approximately 3 to 5) at all times t and then subjected to N-grade time derivative to obtain a higher derivative. Then, biological information such as a heart rate signal is extracted as an amplitude peak position obtained as a result of the higher derivative. Accordingly, the biological information is accurately measured even under a condition with much unnecessary vibration noise.

In JP 2006-264464 A, when a biological sensor unit detects biological information of an occupant of a vehicle, the engine speed is increased. Accordingly, an oscillation frequency of the engine is kept away from an oscillation frequency of the biological information to increase the accuracy in detection of the biological information.

In JP 2006-55504 A, a radio wave Doppler sensor contactlessly applies a radio wave to a body surface and receives a reflected wave from the body surface. Then, an I signal and a Q signal including information of an amplitude component and a phase component of the reflected wave are output. The I signal and the Q signal are subjected to polar coordinate transformation to generate an amplitude component signal and a phase component signal. Further, a motion component of the body surface is separated from both the amplitude component signal and the phase component signal using an independent component analysis technique to extract only an accurate heart rate.

In JP 2010-120493 A, a radio wave unmodulative Doppler sensor detects a movement of a driver of a vehicle. A biological signal of the driver is extracted based on a phase change of an output from the sensor. Further, an estimated distance between the sensor and the driver is calculated based on the integral of a phase change amount of the output from the sensor. Then, a reliability of the biological signal is determined based on the estimated distance. When the reliability is low, the output of the biological signal to an external device is stopped to prevent a reduction in the accuracy of the biological signal.

In JP 2011-15887 A, a Doppler sensor transmits a radio wave to a body surface, and a reflected wave from the body surface is subjected to IQ-detection to sequentially acquire an I signal and a Q signal in a time series manner. Then, biological information is acquired based on the locus of the I signal and the Q signal on an IQ plane.

In JP 10-179527 A, a radio wave is transmitted to a body surface, and a reflected wave from the body surface is converted to an electric signal. Then, phase detection is performed on the electric signal, and an output data row is transmitted to a memory. Further, a specific part of the output data row is selected as a correction data row, and an average offset (correction value) is determined with respect to the correction data row. Then, correction for subtracting the average offset from the output data row is performed, and a data converter performs conversion to blood pressure data. Further, the specific part of the output data row is determined by a signal frequency of the reflected wave to avoid the influence of a phase offset (noise) included in the output data row.

When a radio wave is transmitted to an occupant, a reflected wave from a surrounding object is generated in addition to a reflected wave from the occupant. The reflected wave from the surrounding object is unnecessary for the detection of biological information. Further, a transmission circuit and a reception circuit of a radio wave are affected by temperature changes. Thus, when biological information of an occupant of a vehicle is detected using a radio wave, the accuracy of the detection is reduced by the influence of the surrounding environment such as a surrounding object or temperature.

### SUMMARY

In view of the above, an object of the disclosure is to provide a biological information detection apparatus capable of reducing the influence of the surrounding environment to improve the accuracy in detection of biological information.

A biological information detection apparatus according to one or more embodiments of the disclosure includes: a radio wave transmitter-receiver configured to transmit a radio wave to a body surface of an occupant riding in a vehicle and to receive a reflected wave of the radio wave; a correction value setting unit configured to set a correction value for detecting biological information of the occupant; and a biological information detector configured to detect the biological information of the occupant based on a result of transmitting the radio wave and receiving the reflected wave by the radio wave transmitter-receiver and the correction value set by the correction value setting unit. The biological information detection apparatus further includes: a vehicle opening detector configured to detect that the vehicle is opened so as to allow riding; and a riding detector configured to detect riding of the occupant in the vehicle. The correction value setting unit sets the correction value based on the result of transmitting the radio wave and receiving the reflected wave by the radio wave transmitter-receiver after detection of opening of the vehicle by the vehicle opening detector and before detection of riding of the occupant by the riding detector. Further, the biological information detector detects the biological information based on the result of transmitting the radio wave and receiving the reflected wave by the radio wave transmitter-receiver and the correction value after the detection of riding of the occupant by the riding detector.

According to the above, the correction value setting unit sets the correction value based on the output result affected by the surrounding environment from the radio wave transmitter-receiver after the vehicle is opened so as to allow riding and before the occupant gets in the vehicle, that is, when a radio wave from the radio wave transmitter-receiver is not applied to the occupant. Then, the biological information detector detects the biological information of the occupant based on the output result from the radio wave transmitter-receiver and the correction value set by the correction value setting unit when the occupant rides in the vehicle, that is, when a radio wave from the radio wave transmitter-receiver is applied to the occupant. Thus, it is possible to reduce the influence of the surrounding environment to improve the accuracy in detection of the biological information of the occupant.

In one or more embodiments of the disclosure, in the above biological information detection apparatus, the vehicle opening detector may determine that the vehicle is opened when unlocking or opening of a door from outside of the vehicle is detected.

Further, in one or more embodiments of the disclosure, in the above biological information detection apparatus, the riding detector may determine that the occupant rides in the vehicle when at least one of seating on a predetermined seat of the vehicle, wearing of a seat belt, an operation of a manual operation unit, and starting of a travel driving source is detected.

Furthermore, in one or more embodiments of the disclosure, the above biological information detection apparatus may further include a correction value memory configured to store the correction value every time the correction value setting unit sets the correction value.

According to one or more embodiments of the disclosure, it is possible to provide a biological information detection apparatus capable of reducing the influence of the surrounding environment to improve the accuracy in detection of biological information.

### BRIEF DESCRIPTION OF THE DRAWINGS

FIG. 1 is a configuration diagram of a biological information detection apparatus according to one or more embodiments of the disclosure;

FIG. 2 is a diagram illustrating a vehicle equipped with the biological information detection apparatus of FIG. 1;

FIG. 3 is a detailed diagram of a radio wave transmitter-receiver of FIG. 1;

FIG. 4 is as diagram illustrating data handled in a biological information detector of FIG. 1; and

FIG. 5 is a flowchart illustrating the operation of the biological information detection apparatus of FIG. 1.

### DETAILED DESCRIPTION

Hereinbelow, embodiments of the disclosure will be described with reference to the drawings. Identical reference signs designate identical or corresponding parts throughout the drawings. In embodiments of the disclosure, numerous specific details are set forth in order to provide a more through understanding of the invention. However, it will be apparent to one of ordinary skill in the art that the invention may be practiced without these specific details. In other instances, well-known features have not been described in detail to avoid obscuring the invention.

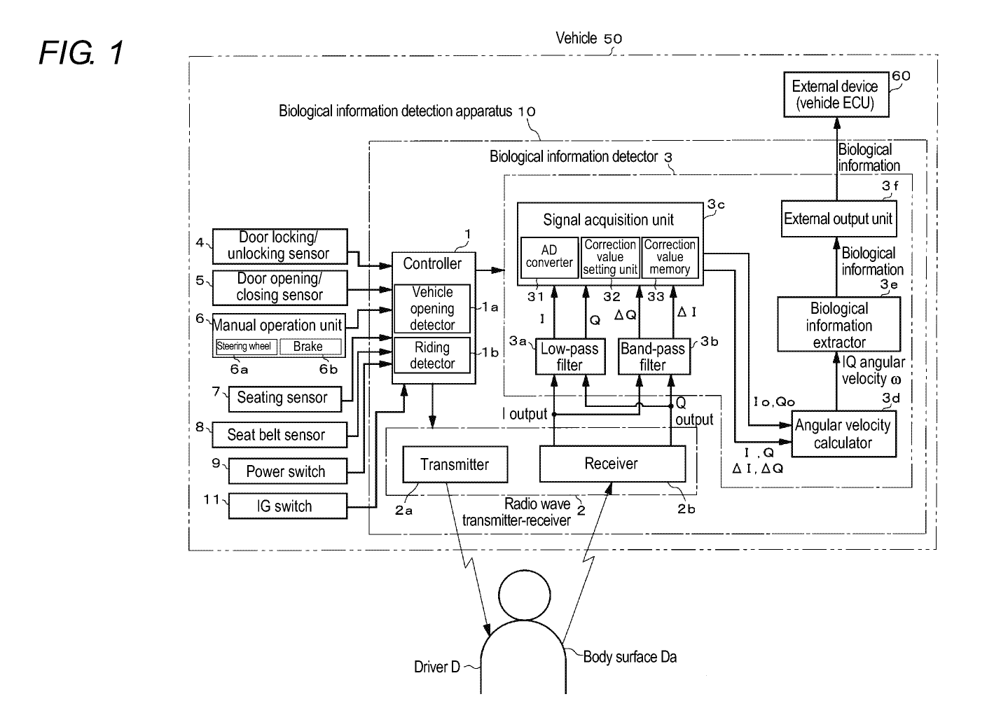

First, the configuration of a biological information detection apparatus 10 of one or more embodiments will be described with reference to FIGS. 1 to 4.

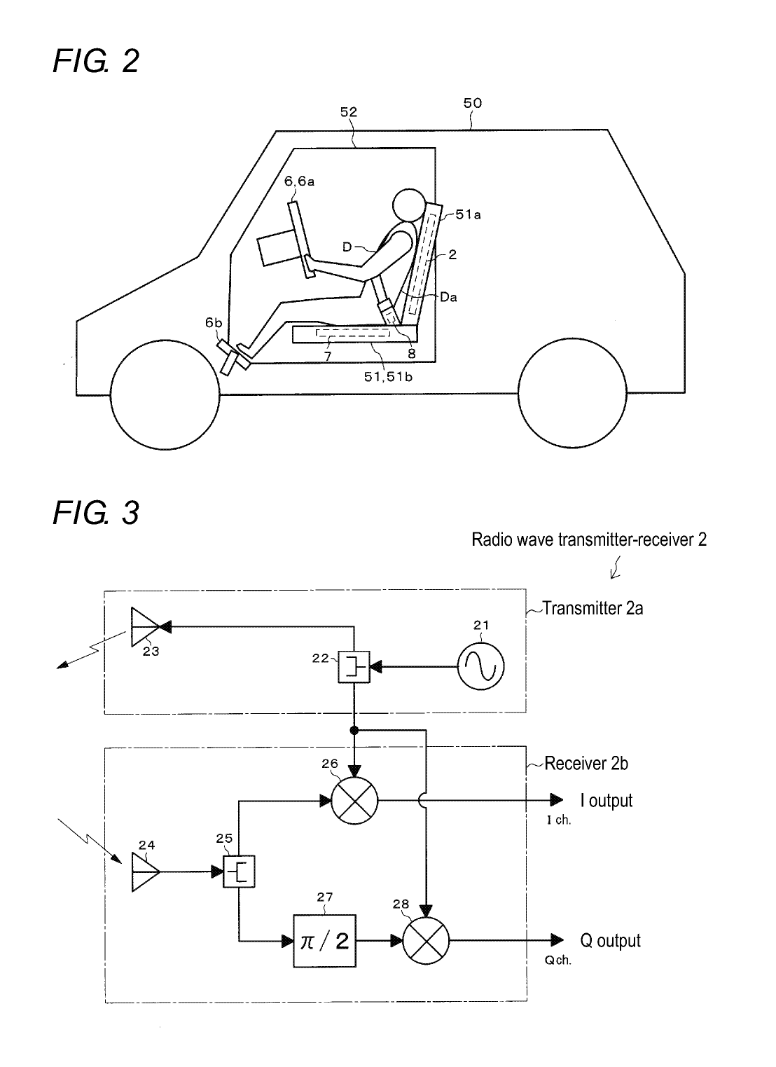

FIG. 1 is a diagram illustrating the configuration of the biological information detection apparatus 10. FIG. 2 is a diagram illustrating a vehicle 50 which is equipped with the biological information detection apparatus 10. FIG. 2 schematically illustrates a state of the vehicle 50 viewed from the lateral side.

The vehicle 50 includes an automatic four-wheel vehicle (FIG. 2). As illustrated in FIG. 1, the vehicle 50 is equipped with the biological information detection apparatus 10, a door locking/unlocking sensor 4, a door opening/closing sensor 5, a manual operation unit 6, a seating sensor 7, a seat belt sensor 8, a power switch 9, and an ignition (IG) switch 11.

The biological information detection apparatus 10 includes a Doppler radar module. The biological information detection apparatus 10 is provided with a controller 1, a radio wave transmitter-receiver 2, and a biological information detector 3. The biological information detection apparatus 10 detects biological information such as a heart rate or a respiration rate of a driver D who is seated on a driver's seat 51 of the vehicle 50 illustrated in FIG. 2.

The controller 1 of FIG. 1 controls the operations of the radio wave transmitter-receiver 2 and the biological information detector 3. The controller 1 is provided with a vehicle opening detector 1a and a riding detector 1b.

As illustrated in FIG. 2, the radio wave transmitter-receiver 2 is embedded in a backrest 51a of the driver's seat 51 of the vehicle 50. As another example, the radio wave transmitter-receiver 2 may be embedded only in a seat portion 51b of the driver's seat 51 or may be embedded in both the seat portion 51b and the backrest 51a. As illustrated in FIG. 1, the radio wave transmitter-receiver 2 is provided with a transmitter 2a and a receiver 2b.

FIG. 3 is a detailed diagram of the radio wave transmitter-receiver 2. The transmitter 2a of the radio wave transmitter-receiver 2 includes a sine wave oscillator 21, a splitter 22, and a transmission antenna 23. The receiver 2b includes a reception antenna 24, a splitter 25, mixers 26, 28, and a π/2 phase shifter 27.

When the sine wave oscillator 21 outputs a sine wave signal, the splitter 22 splits the sine wave signal into two signals. One of the signals is input to the mixer 26 and the mixer 28 of the receiver 2b. The other signal is input to the transmission antenna 23. Accordingly, a radio wave is transmitted to a body surface Da (FIGS. 1 and 2) of the driver D who is seated on the driver's seat 51 of the vehicle 50 from the transmission antenna 23.

The radio wave transmitted from the transmission antenna 23 is reflected by the body surface Da of the driver D, and the reception antenna 24 of the receiver 2b receives the reflected wave. The reflected wave received by the reception antenna 24 is converted into a signal, and the splitter 25 splits the signal into two signals. One of the signals is input to the mixer 26. The other signal is shifted in phase by the π/2 phase shifter 27 so as to be delayed by π/2 (rad) and then input to the mixer 28.

The mixer 26 multiplies the signals input from the splitter 22 and the splitter 25 and outputs a signal from an I channel (I output). The mixer 28 multiplies the signals input from the splitter 22 and the π/2 phase shifter 27 and outputs a signal from a Q channel (Q output).

As illustrated in FIG. 1, the biological information detector 3 is provided with a low-pass filter 3a, a band-pass filter 3b, a signal acquisition unit 3c, an angular velocity calculator 3d, a biological information extractor 3e, and an external output unit 3f.

The I output and the Q output from the receiver 2b of the radio wave transmitter-receiver 2 are allowed to pass through the low-pass filter 3a, so that an I signal and a Q signal which are two IQ orthogonal signals are input to the signal acquisition unit 3c. Further, the I output and the Q output from the receiver 2b are allowed to pass through the band-pass filter 3b, so that a ΔI signal and a ΔQ signal which are obtained by differentiation of the I signal and the Q signal, respectively, are input to the signal acquisition unit 3c (ΔI=dI/dt, ΔQ=dQ/dt).

The signal acquisition unit 3c is provided with an AD converter 31, a correction value setting unit 32, and a correction value memory 33. The AD converter 31 analog-to-digital converts the I signal, the Q signal, the ΔI signal, and the ΔQ signal which are input from the low-pass filter 3a and the band-pass filter 3b. Data items I, Q, ΔI, ΔQ converted by the AD converter 31 are output to the angular velocity calculator 3d.

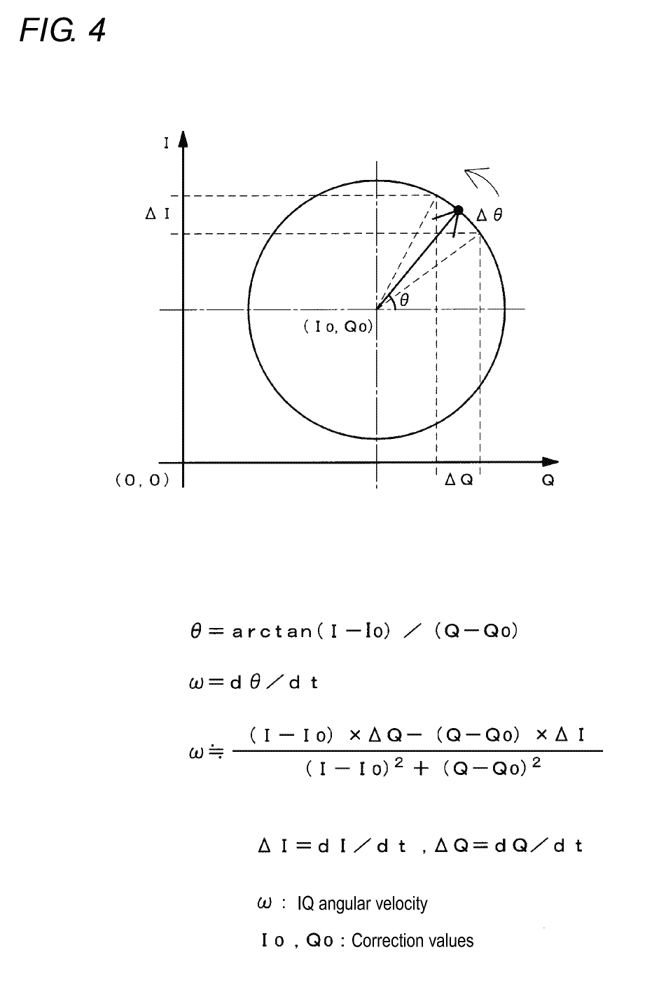

FIG. 4 is a diagram illustrating data handled in the biological information detector 3. In FIG. 4, the vertical axis represents the data item I, and the horizontal axis represents the data item Q. The locus of a plot (black dot) of the data items I, Q is a circle. The size of the circle represents a reception intensity of a reflected wave received by the radio wave transmitter-receiver 2 and varies depending on a state (inclination and reflectance) of the surface of a reflector (e.g., the body surface Da of the driver D). Offset values Io, Qo which are center coordinates of the circle are correction values for detecting biological information of the driver D. The correction value setting unit 32 of the signal acquisition unit 3c sets (calculates) the correction values Io, Qo based on the data items I, Q, ΔI, ΔQ which are obtained by converting the input signals from the low-pass filter 3a and the band-pass filter 3b by the AD converter 31.

Every time the correction value setting unit 32 sets the correction values Io, Qo, the set correction value Io, Qo are stored in the correction value memory 33 to update the contents of the correction value memory 33. Further, the correction values Io, Qo are read from the correction value memory 33 by the correction value setting unit 32 and output to the angular velocity calculator 3d (FIG. 1).

The angular velocity calculator 3d calculates an IQ angular velocity co based on the data items I, Q, ΔI, ΔQ and the correction values Io, Qo which are input from the signal acquisition unit 3c. The IQ angular velocity ω is calculated by the following expression. As illustrated in FIG. 4, θ is an angle of a line segment that connects the plot of the data items I, Q with the offset values Io, Qo and represents a phase.

θ=arctan(I−Io)/(Q−Qo)

ω=dθ/dt

ω≈{(I−Io)×ΔQ−(Q−Qo)×ΔI}/{(I−Io)²+(Q−Qo)²} (1)

The biological information extractor 3e extracts biological information such as a pulse rate or a respiration rate of the driver D based on the IQ angular velocity ω calculated by the angular velocity calculator 3d. The external output unit 3f outputs the biological information extracted by the biological information extractor 3e to an external device 60 such as a vehicle electronic control unit (ECU).

The door locking/unlocking sensor 4 is embedded in a door 52 (FIG. 2) of the driver's seat of the vehicle 50. The door locking/unlocking sensor 4 detects locking and unlocking of the door 52. The vehicle 50 is equipped with a passive entry system, a keyless entry system, or a polling system. In at least one of these systems, locking and unlocking of the door 52 are executed in accordance with wireless communication between an onboard device (not illustrated) and a portable electronic key (not illustrated).

The door opening/closing sensor 5 is embedded in the door 52 or a frame body of the door 52. The door opening/closing sensor 5 detects opening and closing of the door 52.

For example, when the portable electronic key is present outside the vehicle 50, and the door locking/unlocking sensor 4 detects unlocking of the door 52 or the door opening/closing sensor 5 detects opening of the door 52, the vehicle opening detector 1a of the controller 1 of the biological information detection apparatus 10 determines that the vehicle 50 is opened so as to allow riding.

The manual operation unit 6 includes a steering wheel 6a and a brake 6b which are operated by the driver D for manually driving the vehicle 50. When each unit of the manual operation unit 6 is operated, an operation signal thereof is output to the controller 1 of the biological information detection apparatus 10.

The seating sensor 7 detects seating of the driver D on the driver's seat 51 of the vehicle 50. The seat belt sensor 8 detects wearing of a seat belt in the driver's seat of the vehicle 50.

The power switch 9 is operated for starting and stopping an engine which is a travel driving source of the vehicle 50. When the power switch 9 is operated, an operation signal thereof is output to the controller 1 of the biological information detection apparatus 10. The IG switch 11 is turned on when the engine is started. An ON signal of the IG switch 11 is output to the controller 1. When an engine starting operation is performed in the power switch 9 or when the IG switch 11 is turned on, the controller 1 determines that the engine is started. As another example, the travel driving source may include a travel motor.

The riding detector 1b of the controller 1 determines that the driver D rides in the vehicle 50, for example, when receiving at least one of detection of seating on the driver's seat by the seating sensor 7, detection of wearing of the seat belt in the driver's seat by the seat belt sensor 8, an operation of the manual operation unit 6, or starting of the engine.

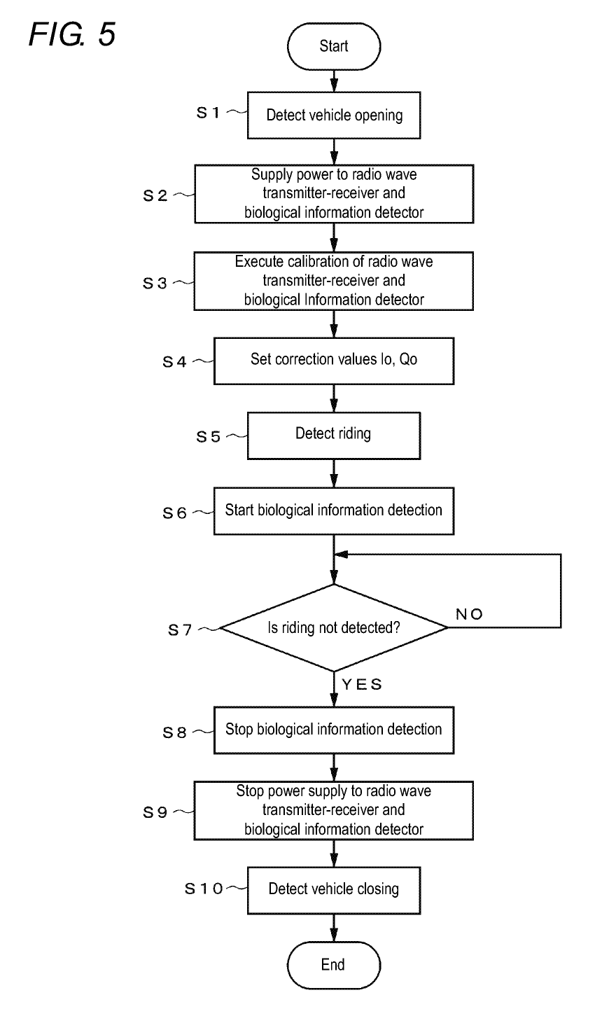

Next, the operation of the biological information detection apparatus 10 will be described with reference to FIG. 5.

FIG. 5 is a flowchart illustrating the operation of the biological information detection apparatus 10.

In a state in which there is no occupant (e.g., the driver D) riding in the vehicle 50, the vehicle opening detector 1a of the biological information detection apparatus 10 detects that the vehicle 50 is opened so as to allow riding from an output result of the door locking/unlocking sensor 4 or the door opening/closing sensor 5 (step S1). Accordingly, the controller 1 supplies power to the radio wave transmitter-receiver 2 and the biological information detector 3 (step S2) and executes calibration of the radio wave transmitter-receiver 2 and the biological information detector 3 (step S3).

In step S3, specifically, the transmitter 2a of the radio wave transmitter-receiver 2 transmits a radio wave, and the receiver 2b receives a reflected wave. At this time, the driver D has not yet got in a vehicle cabin of the vehicle 50. Thus, the radio wave transmitted from the transmitter 2a is not reflected by the body surface Da of the driver D, and the radio wave received by the receiver 2b includes a reflected wave from a surrounding object. Then, as illustrated in FIG. 1, the I output and the Q output are output from the receiver 2b and pass through the low-pass filter 3a and the band-pass filter 3b. Accordingly, the I signal, the Q signal, the ΔI signal, and the ΔQ signal are input to the signal acquisition unit 3c. Further, the AD converter 31 digital-converts the I signal, the Q signal, the ΔI signal, and the ΔQ signal which are thus input to obtain calibration data items I, Q, ΔI, ΔQ.

The calibration for acquiring the above calibration data items I, Q, ΔI, ΔQ is performed from when the vehicle opening detector 1a detects that the vehicle 50 is opened so as to allow riding to when the riding detector 1b detects riding of the driver D in the vehicle 50. Then, when a predetermined number of calibration data items I, Q, ΔI, ΔQ are acquired, the correction value setting unit 32 sets the correction values Io, Qo based on the acquired calibration data items I, Q, ΔI, ΔQ (step S4), and these correction values Io, Qo are stored in the correction value memory 33. At this time, the correction values Io, Qo may be set based on only the calibration data items I, Q.

When the preceding correction values Io, Qo have been stored in the correction value memory 33 at the time of setting the current correction values Io, Qo by the correction value setting unit 32, the preceding correction values Io, Qo are overwritten with the current correction values Io, Qo (updating).

Then, the riding detector 1b detects riding of the driver D in the vehicle 50 from the output result of the manual operation unit 6, the seating sensor 7, the seat belt sensor 8, the power switch 9, or the IG switch 11 (step S5). Accordingly, the controller 1 starts detection of the biological information of the driver D using the radio wave transmitter-receiver 2 and the biological information detector 3 (step S6).

In step S6, specifically, the transmitter 2a of the radio wave transmitter-receiver 2 transmits a radio wave, and the receiver 2b receives a reflected wave. At this time, the driver D gets on the vehicle and is seated on the driver's seat 51. Thus, the radio wave transmitted from the transmitter 2a is reflected by the body surface Da of the driver D, and the receiver 2b receives the reflected wave from the driver D and a reflected wave from a surrounding object. Then, as illustrated in FIG. 1, the I output and the Q output are output from the receiver 2b and pass through the low-pass filter 3a and the band-pass filter 3b. Accordingly, the I signal, the Q signal, the ΔI signal, and the ΔQ signal are input to the signal acquisition unit 3c.

In the signal acquisition unit 3c, the AD converter 31 analog-to-digital converts the I signal, the Q signal, the ΔI signal, and the ΔQ signal which are input thereto, and outputs the converted data items I, Q, ΔI, ΔQ to the angular velocity calculator 3d as needed. Further, the correction value setting unit 32 reads the correction values Io, Qo (fixed values) stored in the correction value memory 33 and outputs the read correction values Io, Qo to the angular velocity calculator 3d.

Then, the angular velocity calculator 3d calculates the IQ angular velocity ω using the above expression (1) based on the input data items I, Q, ΔI, ΔQ and the input correction values Io, Qo. Further, the biological information extractor 3e extracts biological information such as a pulse rate or a respiration rate of the driver D based on the IQ angular velocity ω. Accordingly, biological information with reduced influence of the surrounding environment such as a reflected wave from a surrounding object is obtained. The biological information extracted by the biological information extractor 3e is output to the external device 60 from the external output unit 3f as needed. The external device 60 displays the biological information or utilizes the biological information as control data of the vehicle 50.

The detection of the biological information is performed from when the riding detector 1b detects riding of the driver D in the vehicle 50 to when the riding detector 1b detects that the driver D is not riding in the vehicle 50. That is, while the driver D is riding in the vehicle 50, the above biological information detecting operation is executed regardless of whether the vehicle 50 is in a stopped state or a traveling state.

Then, for example, when the driver D gets off the vehicle 50, the detection of the seating of the driver D on the driver's seat by the seating sensor 7 comes to a stop. Accordingly, the riding detector 1b determines that the driver D is not riding in the vehicle 50. When the detection of the riding of the driver D by the riding detector 1b comes to a stop in this manner (step S7: YES), the controller 1 stops the detection of the biological information of the driver D by the radio wave transmitter-receiver 2 and the biological information detector 3 (step S8) and also stops the power supply to the radio wave transmitter-receiver 2 and the biological information detector 3 (step S9). Then, for example, when the door 52 is closed and locked from the outside of the vehicle 50, the vehicle opening detector 1a detects closing of the vehicle 50 from output results of the door locking/unlocking sensor 4 and the door opening/closing sensor 5 (step S10).

Then, the non-detection of the riding of the driver D in the vehicle 50 in step S7 may be determined based on an element other than the output of the seating sensor 7. For example, the riding detector 1b may determine that the driver D is not riding in the vehicle 50 when receiving at least one of a lapse of a predetermined time or more from an engine stopping operation performed in the power switch 9, a lapse of a predetermined time or more with an off state of the IG switch 11, detection of a release of the seat belt in the driver's seat by the seat belt sensor 8 for a predetermined time or more, or non-detection of seating of the driver D by the seating sensor 7 for a predetermined time or more.

According to an illustrative embodiment, the correction value setting unit 32 sets the correction values Io, Qo based on the output result affected by the surrounding environment from the radio wave transmitter-receiver 2 after the detection of opening of the vehicle 50 by the vehicle opening detector 1a and before the detection of riding of the driver D in the vehicle 50 by the riding detector 1b, that is, when a radio wave from the radio wave transmitter-receiver 2 is not applied to the driver D. Then, the biological information detector 3 detects the biological information of the driver D based on the output result from the radio wave transmitter-receiver 2 and the correction values Io, Qo set by the correction value setting unit 32 after the detection of riding of the driver D in the vehicle 50 by the riding detector 1b, that is, when the driver D rides in the vehicle 50 and a radio wave from the radio wave transmitter-receiver 2 is applied to the driver D. Thus, it is possible to reduce the influence of the surrounding environment to improve the accuracy in detection of the biological information of the driver D.

Further, in an illustrative embodiment, the correction values Io, Qo which are set from when the vehicle 50 is opened so as to allow riding to when the driver D gets in the vehicle 50 are fixed after the driver D gets in the vehicle 50. Then, after the driver D gets in the vehicle 50, the biological information detector 3 detects the biological information based on the correction values Io, Qo and the output result from the radio wave transmitter-receiver 2. Thus, it is not necessary to set and update the correction values Io, Qo every time the biological information detector 3 detects the biological information. As a result, it is possible to reduce processing loads of the correction value setting unit 32 and the biological information detector 3.

Further, in an illustrative embodiment, when the door locking/unlocking sensor 4 or the door opening/closing sensor 5 detects unlocking of the door 52 or opening of the door 52 from the outside of the vehicle 50, the vehicle opening detector 1a determines that the vehicle 50 is opened. Thus, the correction value setting unit 32 can set the correction values Io, Qo based on the output result affected by the surrounding environment from the radio wave transmitter-receiver 2 immediately before the driver D gets in the vehicle 50.

Further, in an illustrative embodiment, the riding detector 1b determines that the driver D rides in the vehicle 50 when at least one of seating of the driver D on the driver's seat of the vehicle 50, wearing of the seat belt, an operation of the manual operation unit 6, and starting of the engine is detected. Thus, it is possible to reliably detect that the driver D gets onto the driver's seat of the vehicle 50 so that the radio wave transmitter-receiver 2 can transmit and receive a radio wave to and from the driver D.

Further, in an illustrative embodiment, every time the correction value setting unit 32 sets the correction values Io, Qo, the set correction values Io, Qo are stored in the correction value memory 33 to update the contents of the correction value memory 33. Thus, it is possible to record the latest correction values Io, Qo taking the surrounding environment into consideration in the correction value memory 33 every time the driver D gets in the vehicle 50. Further, it is possible to accurately detect the biological information of the driver D based on the output result from the radio wave transmitter-receiver 2 and the latest correction values Io, Qo recorded in the correction value memory 33 after the driver D gets in the vehicle 50.

In one or more embodiments of the disclosure, various embodiments other than an illustrative embodiment can be employed. For example, in the example of illustrative embodiments, the detection of the biological information of the driver D is stopped when the detection of riding of the driver D in the vehicle 50 comes to a stop. However, the disclosure is not limited only thereto. Alternatively, for example, the detection of the biological information of the driver D may be stopped when it is determined that the driver D has no intention of driving the vehicle 50 due to an engine stopping operation performed by the driver D using the power switch 9 or a lapse of a predetermined time or more with no operation of the manual operation unit 6.

Further, the condition for detecting opening of the vehicle 50 and the condition for detecting riding in the vehicle 50 are not limited to the conditions described in illustrative embodiments, and other conditions may be used. For example, the position of the portable electronic key inside and outside of the vehicle may be determined by wireless communication between the onboard device and the portable electronic key and may be used as the condition for detecting opening of the vehicle 50 and the condition for detecting riding in the vehicle 50.

Further, in example of illustrative embodiments, the disclosure is applied to the biological information detection apparatus 10 that detects the biological information such as a pulse rate or a respiration rate of the driver D of the vehicle 50. Further, the disclosure can also be applied to a biological information detection apparatus that detects biological information of an occupant other than the driver or a biological information detection apparatus that detects other biological information such as a heart rate or a blood pressure.

While the invention has been described with reference to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

1. A biological information detection apparatus comprising:
a radio wave transmitter-receiver configured to transmit a radio wave to a body surface of an occupant riding in a vehicle and to receive a reflected wave of the radio wave;
a correction value setting unit configured to set a correction value for detecting biological information of the occupant;
a biological information detector configured to detect the biological information of the occupant based on a result of transmitting the radio wave and receiving the reflected wave by the radio wave transmitter-receiver and the correction value set by the correction value setting unit;
a vehicle opening detector configured to detect that the vehicle is opened so as to allow riding; and
a riding detector configured to detect riding of the occupant in the vehicle,
wherein the correction value setting unit sets the correction value based on the result of transmitting the radio wave and receiving the reflected wave by the radio wave transmitter-receiver after detection of opening of the vehicle by the vehicle opening detector and before detection of riding of the occupant by the riding detector, and
wherein the biological information detector detects the biological information based on the result of transmitting the radio wave and receiving the reflected wave by the radio wave transmitter-receiver and the correction value after the detection of riding of the occupant by the riding detector.

2. The biological information detection apparatus according to claim 1,
wherein the vehicle opening detector determines that the vehicle is opened when unlocking or opening of a door from outside of the vehicle is detected.

3. The biological information detection apparatus according to claim 1,
wherein the riding detector determines that the occupant rides in the vehicle when at least one of seating on a predetermined seat of the vehicle, wearing of a seat belt, an operation of a manual operation unit, and starting of a travel driving source is detected.

4. The biological information detection apparatus according to claim 1, further comprising a correction value memory configured to store the correction value every time the correction value setting unit sets the correction value.
